(19) United States
(12) Patent Application Publication
(10) Pub. No.: US 2022/0087583 A1
(43) Pub. Date: Mar. 24, 2022

(54) EVALUATION DEVICE, EVALUATION METHOD, AND EVALUATION PROGRAM

(71) Applicant: JVCKENWOOD Corporation, Yokohama-shi (JP)

(72) Inventor: Takahiro Hayashi, Yokohama-shi (JP)

(21) Appl. No.: 17/543,849

(22) Filed: Dec. 7, 2021

Related U.S. Application Data
(63) Continuation of application No. PCT/JP2020/024119, filed on Jun. 19, 2020.

(30) Foreign Application Priority Data
Jun. 19, 2019 (JP) 2019-113412

(51) Int. Cl. A61B 5/16; A61B 5/00; G16H 50/30

(52) U.S. Cl. CPC A61B 5/163; A61B 5/4088; G16H 50/30; A61B 5/742

(57) ABSTRACT

An evaluation device includes: a display; a gaze point detection unit detecting a position of a subject's gaze point; a display control unit displaying, after displaying a question image, an answer image including a specific object and comparison objects, and when the question image is displayed, displaying a reference image illustrating a positional relationship between the specific object and the comparison objects in the answer image; an area setting unit setting, on the display, a specific area corresponding to the specific object and comparison areas corresponding to the comparison objects; a determination unit determining, at each specified determination cycle, in which area the gaze point is present among the specific area and the comparison areas, based on a position of the gaze point; a calculation unit calculating an evaluation parameter based on a determination result; and an evaluation unit obtaining evaluation data on the subject based on the evaluation parameter.

### CROSS-REFERENCE TO RELATED APPLICATION

This application is a Continuation of PCT International Application No. PCT/JP2020/024119 filed on Jun. 19, 2020 which claims the benefit of priority from Japanese Patent Application No. 2019-113412 filed on Jun. 19, 2019, the entire contents of both of which are incorporated herein by reference.

### BACKGROUND OF THE INVENTION

### 1. Field of the Invention

The present disclosure relates to an evaluation device, an evaluation method, and an evaluation program.

### 2. Description of the Related Art

In recent years, it has been said that cognitive dysfunction and brain dysfunction are on the increase, and there is a need to detect such cognitive dysfunction and brain dysfunction at an early stage and quantitatively evaluate the severity of symptoms. It is known that symptoms of cognitive dysfunction and brain dysfunction affect cognitive ability. For this reason, the subject is evaluated based on the cognitive ability of the subject. For example, there is the disclosure of a device that displays a plurality of types of numbers, prompts the subject to add the numbers and give an answer, and checks the answer given by the subject (see, for example, JP 2011-083403 A).

However, in the method of JP 2011-083403 A and the like, the subject selects an answer by operating a touch panel or the like, and it is difficult to obtain high evaluation accuracy due to an accidental correct answer or an operation error of the subject. For that reason, there is a need to accurately evaluate cognitive dysfunction and brain dysfunction.

### SUMMARY OF THE INVENTION

An evaluation device according to the present disclosure includes a display unit, a gaze point detection unit, a display control unit, an area setting unit, a determination unit, a calculation unit, and an evaluation unit. The gaze point detection unit is configured to detect a position of a gaze point of a subject on the display unit. The display control unit is configured to, after displaying a question image including question information for the subject on the display unit, display, on the display unit, an answer image including a specific object that is a correct answer to the question information and one or more comparison objects different from the specific object, and when the question image is displayed on the display unit, display, on the display unit, a reference image illustrating a positional relationship between the specific object and the one or more comparison objects in the answer image. The area setting unit is configured to set, on the display unit, a specific area corresponding to the specific object and one or more comparison areas corresponding to the one or more comparison objects. The determination unit is configured to determine, at each specified determination cycle, in which area the gaze point is present among the specific area and the one or more comparison areas, based on a position of the gaze point. The calculation unit is configured to calculate an evaluation parameter based on a determination result of the determination unit. The evaluation unit is configured to obtain evaluation data on the subject based on the evaluation parameter.

An evaluation method according to the present disclosure includes: detecting a position of a gaze point of a subject on a display unit; after displaying a question image including question information for the subject on the display unit, displaying, on the display unit, an answer image including a specific object that is a correct answer to the question information and one or more comparison objects different from the specific object, and when the question image is displayed on the display unit, displaying, on the display unit, a reference image illustrating a positional relationship between the specific object and the one or more comparison objects in the answer image; setting, on the display unit, a specific area corresponding to the specific object and one or more comparison areas corresponding to the one or more comparison objects; determining, at each specified determination cycle, in which area the gaze point is present among the specific area and the one or more comparison areas, based on a position of the gaze point; calculating an evaluation parameter based on a determination result obtained at the determining; and obtaining evaluation data on the subject based on the evaluation parameter.

A non-transitory computer-readable recording medium according to the present disclosure contains a computer program. The computer program causes a computer to execute: detecting a position of a gaze point of a subject on a display unit; after displaying a question image including question information for the subject on the display unit, displaying, on the display unit, an answer image including a specific object that is a correct answer to the question information and one or more comparison objects different from the specific object, and when the question image is displayed on the display unit, displaying, on the display unit, a reference image illustrating a positional relationship between the specific object and the one or more comparison objects in the answer image; setting, on the display unit, a specific area corresponding to the specific object and one or more comparison areas corresponding to the one or more comparison objects; determining, at each specified determination cycle, in which area the gaze point is present among the specific area and the one or more comparison areas, based on a position of the gaze point; calculating an evaluation parameter based on a determination result obtained at the determining; and obtaining evaluation data on the subject based on the evaluation parameter.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram schematically illustrating an example of an evaluation device according to the present embodiment;

FIG. 2 is a functional block diagram illustrating an example of the evaluation device;

FIG. 3 is a diagram illustrating an example of a question image displayed on a display unit;

FIG. 4 is a diagram illustrating an example of an intermediate image displayed on the display unit;

FIG. 5 is a diagram illustrating another example of the intermediate image displayed on the display unit;

FIG. 6 is a diagram illustrating an example of an answer image displayed on the display unit;

FIG. 7 is a diagram illustrating an example of a case where an eye-catching video is displayed on the display unit;

FIG. 8 is a flowchart illustrating an example of an evaluation method according to the present embodiment;

FIG. 9 is a diagram illustrating another example of the intermediate image displayed on the display unit; and

FIG. 10 is a flowchart illustrating another example of the evaluation method according to the present embodiment.

### DETAILED DESCRIPTION OF THE PREFERRED EMBODIMENTS

An embodiment of an evaluation device, an evaluation method, and an evaluation program according to the present disclosure are described below with reference to the drawings. The present invention is not limited to the embodiment. Components in the embodiment described below include those that may be easily replaced by those skilled in the art or those that are substantially identical.

In the description below, a three-dimensional global coordinate system is set to describe the positional relationship of units. A direction parallel to a first axis of a predetermined plane is an X-axis direction, a direction parallel to a second axis of the predetermined plane perpendicular to the first axis is a Y-axis direction, and a direction parallel to a third axis perpendicular to both the first axis and the second axis is a Z-axis direction. The predetermined plane includes an XY plane.

Evaluation Device

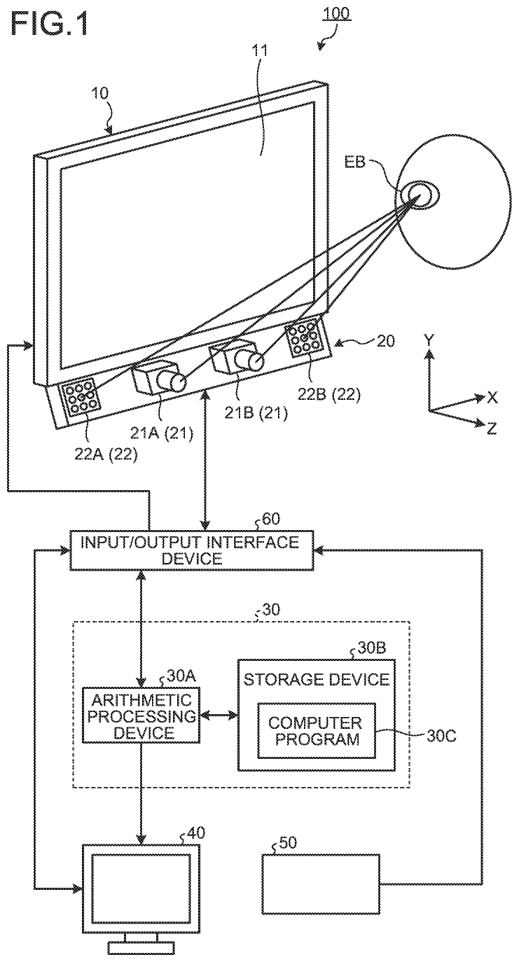

FIG. 1 is a diagram schematically illustrating an example of an evaluation device 100 according to the present embodiment. The evaluation device 100 according to the present embodiment detects the line of sight of a subject and uses a detection result to evaluate cognitive dysfunction and brain dysfunction. The evaluation device 100 may detect the line of sight of the subject by using various methods, such as a method for detecting the line of sight based on the position of the pupil of the subject and the position of a corneal reflection image, or a method for detecting the line of sight based on the position of the inner corner of the eye of the subject and the position of the iris.

As illustrated in FIG. 1, the evaluation device 100 includes a display device 10, an image acquisition device 20, a computer system 30, an output device 40, an input device 50, and an input/output interface device 60. The display device 10, the image acquisition device 20, the computer system 30, the output device 40, and the input device 50 perform data communications via the input/output interface device 60. The display device 10 and the image acquisition device 20 each include a drive circuit that is not illustrated.

The display device 10 includes a flat panel display such as a liquid crystal display (LCD) or an organic electroluminescence display (OLED). According to the present embodiment, the display device 10 includes a display unit 11. The display unit 11 displays information such as an image. The display unit 11 is substantially parallel to the XY plane. The X-axis direction is a horizontal direction of the display unit 11, the Y-axis direction is a vertical direction of the display unit 11, and the Z-axis direction is a depth direction perpendicular to the display unit 11. The display device 10 may be a head-mounted display device. When the display device 10 is a head-mounted display device, a configuration such as the image acquisition device 20 is provided in a head-mounted module.

The image acquisition device 20 acquires image data of right and left eyeballs EB of the subject and transmits the acquired image data to the computer system 30. The image acquisition device 20 includes an image capturing device 21. The image capturing device 21 captures the right and left eyeballs EB of the subject to acquire image data. The image capturing device 21 includes various cameras corresponding to a method for detecting the line of sight of the subject. For example, in the case of the method for detecting the line of sight based on the position of the pupil of the subject and the position of the corneal reflection image, the image capturing device 21 includes an infrared camera, an optical system that allows transmission of near-infrared light having a wavelength of, for example, 850 (nm), and an imaging element capable of receiving the near-infrared light. For example, in the case of the method for detecting the line of sight based on the position of the inner corner of the eye of the subject and the position of the iris, the image capturing device 21 includes a visible light camera. The image capturing device 21 outputs a frame synchronization signal. The cycle of frame synchronization signals may be, for example, but is not limited thereto, 20 (msec). The image capturing device 21 may be configured as, but is not limited thereto, a stereo camera including, for example, a first camera 21A and a second camera 21B.

Further, in the case of a method for detecting the line of sight based on, for example, the position of the pupil of the subject and the position of the corneal reflection image, the image acquisition device 20 includes a lighting device 22 that illuminates the eyeball EB of the subject. The lighting device 22 includes a light emitting diode (LED) light source and may emit near-infrared light having a wavelength of, for example, 850 (nm). In the case of a method for detecting the line of sight based on, for example, the position of the inner corner of the eye of the subject and the position of the iris, the lighting device 22 may be omitted. The lighting device 22 emits a detection light so as to synchronize with the frame synchronization signal of the image capturing device 21. The lighting device 22 may be configured to include, for example, but is not limited thereto, a first light source 22A and a second light source 22B.

The computer system 30 comprehensively controls an operation of the evaluation device 100. The computer system 30 includes an arithmetic processing device 30A and a storage device 30B. The arithmetic processing device 30A includes a microprocessor such as a central processing unit (CPU). The storage device 30B includes a memory or storage such as a read only memory (ROM) and a random access memory (RAM). The arithmetic processing device 30A performs arithmetic processing in accordance with a computer program 30C stored in the storage device 30B.

The output device 40 includes a display device such as a flat panel display. The output device 40 may include a printing device. The display device 10 may also serve as the output device 40. The input device 50 is operated to generate input data. The input device 50 includes a keyboard or mouse for a computer system. The input device 50 may include a touch sensor provided on the display unit of the output device 40, which is a display device.

In the evaluation device 100 according to the present embodiment, the display device 10 and the computer system 30 are separate devices. The display device 10 and the computer system 30 may be integrated. For example, the evaluation device 100 may include a tablet-type personal computer. In this case, the tablet-type personal computer may be equipped with a display device, an image acquisition device, a computer system, an input device, an output device, etc.

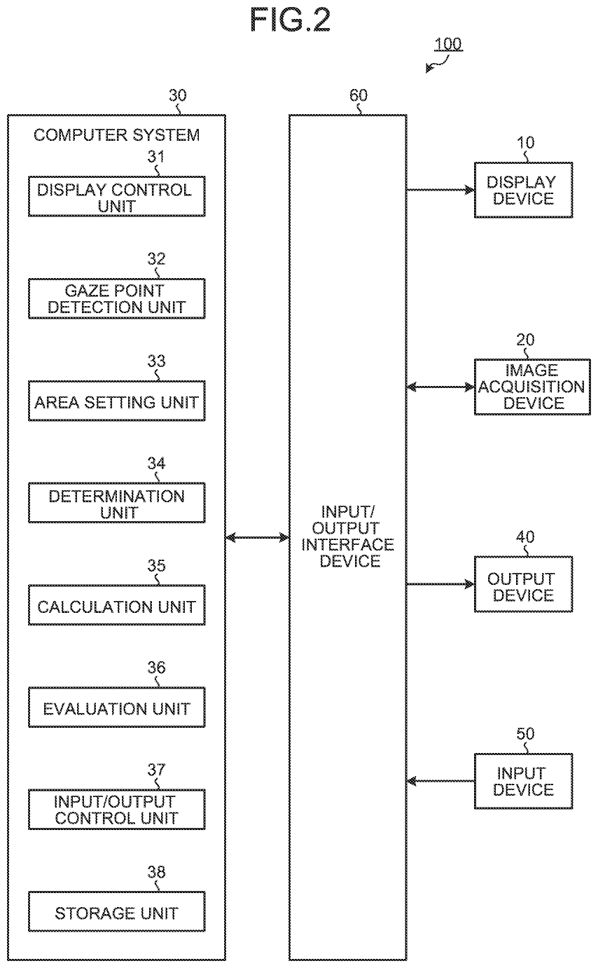

FIG. 2 is a functional block diagram illustrating an example of the evaluation device 100. As illustrated in FIG. 2, the computer system 30 includes a display control unit 31, a gaze point detection unit 32, an area setting unit 33, a determination unit 34, a calculation unit 35, an evaluation unit 36, an input/output control unit 37, and a storage unit 38. The arithmetic processing device 30A and the storage device 30B (see FIG. 1) perform the functions of the computer system 30. Some functions of the computer system 30 may be provided outside the evaluation device 100.

The display control unit 31 displays a question image including question information for the subject on the display unit 11. After displaying the question image on the display unit 11, the display control unit 31 displays, on the display unit 11, an answer image including a specific object that is a correct answer to the question information and one or more comparison objects different from the specific object. When the question image is displayed on the display unit 11, the display control unit 31 displays a reference image illustrating the positional relationship between the specific object and the one or more comparison objects in the answer image as part of the question image. The reference image includes a first object corresponding to the specific object in the answer image and one or more second objects corresponding to the one or more comparison objects in the answer image. The first object and the one or more second objects are arranged so as to have the same positional relationship as that of the specific object and the comparison objects. For example, an image obtained by increasing the transmissivity of the answer image or an image obtained by reducing the size of the answer image may be used as the reference image.

The display control unit 31 displays the reference image on the display unit 11 after the elapse of a predetermined time from the start of display of the question image. For example, the display control unit 31 may display the reference image so as to be superimposed on the question information or may display the reference image at a position away from the question information.

The question image, the answer image, and an intermediate image in which the question image includes the reference image may be previously generated. In this case, the display control unit 31 may switch three images, for example, after displaying the question image, displays the intermediate image after the elapse of a predetermined time, and displays the answer image after the elapse of a predetermined time from the display of the intermediate image.

The gaze point detection unit 32 detects position data on the gaze point of the subject. According to the present embodiment, the gaze point detection unit 32 detects the subject's line-of-sight vector defined by the three-dimensional global coordinate system based on the image data of the right and left eyeballs EB of the subject acquired by the image acquisition device 20. The gaze point detection unit 32 detects the position data on the intersection between the detected line-of-sight vector of the subject and the display unit 11 of the display device 10 as position data on the gaze point of the subject. Specifically, according to the present embodiment, the position data on the gaze point is the position data on the intersection between the line-of-sight vector of the subject defined by the three-dimensional global coordinate system and the display unit 11 of the display device 10. The gaze point detection unit 32 detects the position data on the gaze point of the subject at each specified sampling cycle. The sampling cycle may be, for example, the cycle (e.g., every 20 (msec)) of the frame synchronization signal output from the image capturing device 21.

The area setting unit 33 sets, on the display unit 11, the specific area corresponding to the specific object displayed in the answer image and the comparison areas respectively corresponding to the comparison objects. The area setting unit 33 also sets, on the display unit 11, reference areas corresponding to the reference image displayed in the question image. In this case, the area setting unit 33 may set a first reference area corresponding to the specific object in the reference image and one or more second reference areas respectively corresponding to one or more comparison objects in the reference image.

In the period during which the area setting unit 33 sets the specific area and the comparison areas, the determination unit 34 determines in which area the gaze point is present among the specific area and the comparison areas based on the position data on the gaze point and outputs determination result as determination data. Further, in the period during which the area setting unit 33 sets the reference areas, the determination unit 34 determines in which reference area the gaze point is present among the reference areas (the first reference area and the second reference areas) based on the position data on the gaze point and outputs a determination result as determination data. The determination unit 34 determines in which area the gaze point is present among the specific area and the comparison area at each specified determination cycle. The determination unit 34 also determines in which reference area the gaze point is present among the reference areas at each specified determination cycle. The determination cycle may be, for example, the cycle (e.g., every 20 (msec)) of the frame synchronization signal output from the image capturing device 21. That is, the determination cycle of the determination unit 34 is the same as the sampling cycle of the gaze point detection unit 32. The determination unit 34 makes a determination regarding the gaze point every time the position of the gaze point is sampled by the gaze point detection unit 32, and outputs determination data.

The calculation unit 35 calculates, based on the determination data of the determination unit 34, an evaluation parameter indicating the course of movement of the gaze point in the period during which the specific area and the comparison areas described above are set. The calculation unit 35 also calculates, based on the determination data of the determination unit 34, the evaluation parameter indicating the course of movement of the gaze point in the period during which the reference areas (the first reference area and the second reference areas) described above are set. According to the present embodiment, the gaze point is included in a designated point that is designated by the subject on the display unit.

As the evaluation parameters, the calculation unit 35 calculates at least one piece of data among, for example, arrival time data, movement number data, and presence time data and last area data. In the period during which the specific area and the comparison areas are set, the arrival time data indicates the time until an arrival time when the gaze point first arrives at the specific area. The movement number data indicates the number of times the position of the gaze point moves between the comparison areas before the gaze point first arrives at the specific area. The presence time data indicates the presence time during which the gaze point is present in the specific area in the display period of the reference image. The last area data indicates the area where the gaze point is last present in the display period among the specific area and the comparison areas. In the period during which the reference areas (the first reference area and the second reference areas) are set, the arrival time data indicates the time until an arrival time when the gaze point first arrives at the first reference area. The movement number data indicates the number of times the position of the gaze point moves between the second reference areas before the gaze point first arrives at the first reference area. The presence time data indicates the presence time during which the gaze point is present in the first reference area in the display period of the reference image. The last area data indicates the area where the gaze point is last present in the display period among the first reference area and the second reference areas.

The calculation unit 35 includes a timer that detects the elapsed time after the display unit 11 displays an evaluation video, and a counter that counts the number of times the determination unit 34 determines that the gaze point is present each in the specific area, the comparison area, and the reference areas (the first reference area and the second reference areas). The calculation unit 35 may include a management timer that manages the play time of the evaluation video.

The evaluation unit 36 obtains evaluation data on the subject based on the evaluation parameter. The evaluation data includes the data for evaluating whether the subject is able to gaze at the specific object and the comparison objects displayed on the display unit 11.

The input/output control unit 37 acquires data (image data of the eyeball EB, input data, etc.) from at least either one of the image acquisition device 20 and the input device 50. The input/output control unit 37 outputs data to at least either one of the display device 10 and the output device 40. The input/output control unit 37 may output a task for the subject from the output device 40 such as a speaker. When an answer pattern is displayed a plurality of times in succession, the input/output control unit 37 may output an instruction for causing the subject to gaze at the specific object again from the output device 40 such as a speaker.

The storage unit 38 stores therein the determination data, the evaluation parameters (the arrival time data, the movement number data, the presence time data, and the last area data) and the evaluation data described above. The storage unit 38 stores therein an evaluation program causing the computer to execute: detecting the position of the gaze point of the subject on the display unit 11; after displaying the question image including the question information for the subject on the display unit 11, displaying, on the display unit 11, the answer image including the specific object that is a correct answer to the question information and the comparison objects different from the specific object, and when the question image is displayed on the display unit 11, displaying, on the question image, the reference image illustrating the positional relationship between the specific object and the comparison objects in the answer image; setting, on the display unit 11, the specific area corresponding to the specific object and the comparison areas corresponding to the comparison objects; determining, at each specified determination cycle, in which area the gaze point is present among the specific area and the comparison areas, based on the position of the gaze point; calculating the evaluation parameter based on a determination result of the determination unit 34; and obtaining the evaluation data on the subject based on the evaluation parameter.

Evaluation Method

Next, an evaluation method according to the present embodiment is described. With the evaluation method according to the present embodiment, cognitive dysfunction and brain dysfunction of the subject are evaluated by using the evaluation device 100 described above.

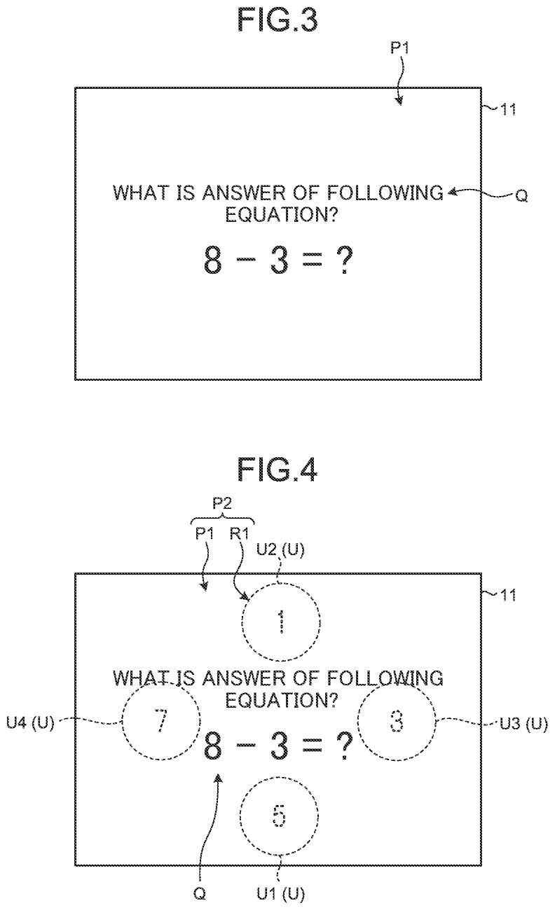

FIG. 3 is a diagram illustrating an example of the question image displayed on the display unit 11. As illustrated in FIG. 3, the display control unit 31 displays, for example, a question image P1 including question information Q for the subject on the display unit 11 for a predetermined period. In the present embodiment, the question information Q is described as an example of the question having the content prompting the subject to calculate the answer of the subtraction “8-3=?”. The question information Q is not limited to the content prompting the subject to calculate and may be a question having other content. In addition to displaying the question information Q, the input/output control unit 37 may output the sound corresponding to the question information Q from the speaker.

FIG. 4 is a diagram illustrating an example of the reference image displayed on the display unit 11. As illustrated in FIG. 4, when the question image P1 is displayed, the display control unit 31 may display a reference image R1 on the display unit 11 at the same time as the question image P1. Hereinafter, the question image P1 in which the reference image is displayed is referred to as an intermediate image P2. For example, the intermediate image P2 in which the question image P1 includes the reference image R1 is previously generated. In this case, the display control unit 31 displays the question image P1 and then, after the elapse of a predetermined time, displays the intermediate image P2. In the intermediate image P2 illustrated in FIG. 4, the reference image R1 is, for example, an image obtained by increasing the transmissivity of an answer image P3 described below. The display control unit 31 may display the reference image R1 in a superimposed manner on the question image P1. The display control unit 31 may display the intermediate image P2 including the reference image R1 after the elapse of a predetermined time from the start of display of the question image P1.

The reference image R1 includes reference objects U. The reference objects U include a first object U1 and second objects U2, U3, and U4. The first object U1 corresponds to a specific object M1 (see FIG. 6) in the answer image P3. The second objects U2 to U4 correspond to comparison objects M2 to M4 (see FIG. 6) in the answer image P3. The first object U1 and the second objects U2 to U4 are arranged so as to have the same positional relationship as that of the specific object M1 and the comparison objects M2 to M4 (see FIG. 6) in the answer image P3.

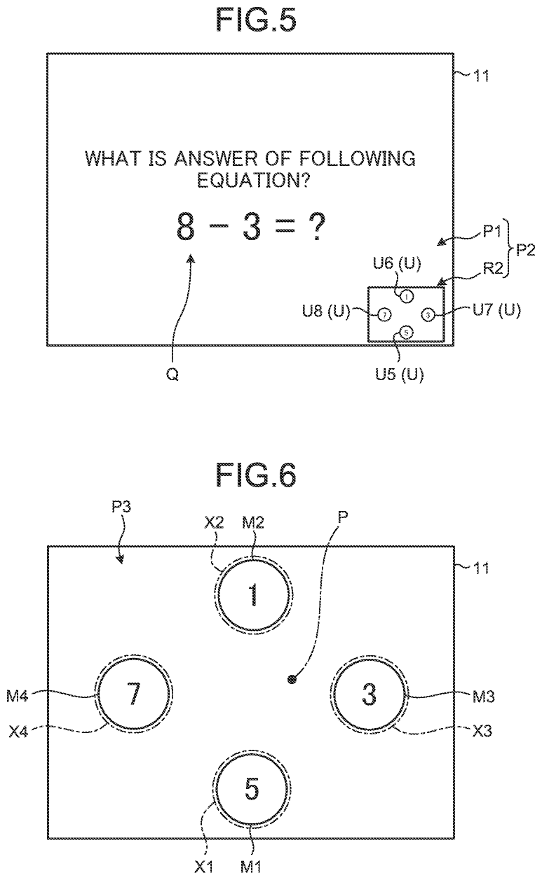

FIG. 5 is a diagram illustrating another example of the intermediate image displayed on the display unit 11. The intermediate image P2 illustrated in FIG. 5 includes a reference image R2 as part of the question image P1. The reference image R2 is, for example, an image obtained by reducing the size of the answer image P3 described below. The reference image R2 is displayed at a position that is not overlapped with the question information Q, i.e., a position outside the display area of the question information Q in the display unit 11, such as a corner portion of the display unit 11. The reference image R2 may be arranged at another position different from the corner portion of the display unit 11 as long as the reference image R2 is not overlapped with the question information Q.

The reference image R2 includes the reference objects U. The reference objects U include a first object U5 and second objects U6, U7, and U8. The first object U5 corresponds to the specific object M1 (see FIG. 6) in the answer image P3. The second objects U6 to U8 correspond to the comparison objects M2 to M4 (see FIG. 6) in the answer image P3. The first object U5 and the second objects U6 to U8 are arranged so as to have the same positional relationship as that of the specific object M1 and the comparison objects M2 to M4 (see FIG. 6) in the answer image P3.

FIG. 6 is a diagram illustrating an example of the answer image displayed on the display unit 11. As illustrated in FIG. 6, the display control unit 31 displays the answer image P3 on the display unit 11 after the elapse of a predetermined time from the display of the intermediate image P2. Although FIG. 6 illustrates an example of a gaze point P that is displayed as a result for example after a measurement in the display unit 11, the gaze point P is not actually displayed in the display unit 11. The answer image P3 includes the specific object M1 that is a correct answer to the question information Q and the comparison objects M2 to M4 that are incorrect answers to the question information Q. The specific object M1 is the number “5” that is a correct answer to the question information Q. The comparison objects M2 to M4 are the numbers “1”, “3”, and “7” that are incorrect answers to the question information Q.

The area setting unit 33 sets a specific area X1 corresponding to the specific object M1, which is a correct answer to the question information Q, in the period during which the answer image P3 is displayed. The area setting unit 33 sets comparison areas X2 to X4 corresponding to the comparison objects M2 to M4, which are incorrect answers to the question information Q.

The area setting unit 33 may set the specific area X1 and the comparison areas X2 to X4 in respective areas including at least parts of the specific object M1 and the comparison objects M2 to M4. In the present embodiment, the area setting unit 33 sets the specific area X1 in the circular area including the specific object M1 and sets the comparison areas X2 to X4 in the circular areas including the comparison objects M2 to M4.

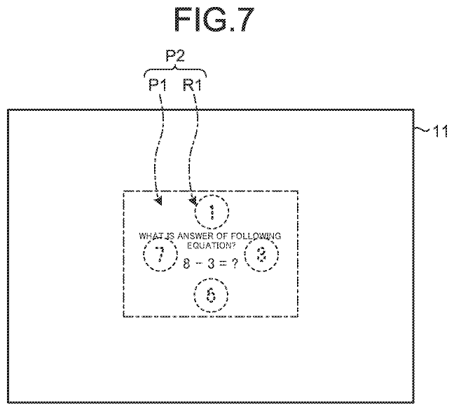

FIG. 7 is a diagram illustrating an example of a case where an eye-catching video is displayed on the display unit 11. When the display of the intermediate image P2 is switched to the display of the answer image P3, the display control unit 31 may display, as an eye-catching video, a video obtained by reducing the intermediate image P2 toward a target position such as a central portion of the display unit 11 on the display unit 11, as illustrated in FIG. 7. In this case, the display control unit 31 also reduces the reference image R1 (or the reference image R2) displayed on the intermediate image P2 as an image integrated with the intermediate image P2. Accordingly, the line of sight of the subject may be guided to the target position.

It is known that the symptoms of cognitive dysfunction and brain dysfunction affect the cognitive ability and calculation ability of the subject. When the subject does not have cognitive dysfunction and brain dysfunction, the subject may recognize the question information Q and do a calculation in the question image P1 and may gaze at the specific object M1, which is a correct answer, in the answer image P3. When the subject has cognitive dysfunction and brain dysfunction, the subject may fail to recognize the question information Q as well as well as do a calculation in the question image P1 and may fail to gaze at the specific object M1, which is a correct answer, in the answer image P3.

In the case of the display described above, how the specific object M1 and the comparison objects M2 to M4 are arranged in the answer image P3 is unknown until the answer image P3 is displayed. Therefore, when the display of the answer image P3 is started, the subject needs to see the entire display unit 11 to understand how the specific object M1 and the comparison objects M2 to M4 are arranged. This action may reduce the accuracy even for the subject having no cognitive dysfunction and brain dysfunction to evaluate the process from when the display of the answer image P3 is started until when the specific object M1 is gazed at.

In a method in which the specific object M1 and the comparison objects M2 to M4 are simply displayed on the display unit 11 to be gazed at, the gaze point of the subject may be accidentally placed on the specific object M1, which is a correct answer, during the display period of the answer image P3. In such a case, the correct answer may be determined regardless of whether the subject has cognitive dysfunction and brain dysfunction, and therefore it is difficult to evaluate the subject with high accuracy.

Therefore, for example, the following procedure is executed, whereby the subject can be evaluate with high accuracy. First, the display control unit 31 displays the question image P1 on the display unit 11. After the elapse of a predetermined time from the start of display of the question image P1, the display control unit 31 displays the intermediate image P2 in which the question image P1 includes the reference image R1 (or R2). The reference image R1 illustrates the arrangement of the specific object M1 and the comparison objects M2 to M4 in the subsequently displayed answer image P3. The display control unit 31 displays the answer image P3 on the display unit 11 after the elapse of a predetermined time from the display of the intermediate image P2.

By the execution of this procedure, to answer the question information Q displayed on the question image P1, the subject gazes at the reference image R1 in the intermediate image P2 before the answer image P3 is displayed so as to understand the arrangement of the specific object M1 and the comparison objects M2 to M4. This allows the subject to quickly gaze at the specific object M1, which is a correct answer to the question information Q, after the answer image P3 is displayed.

The gaze point detection unit 32 detects the position data on the gaze point P of the subject at each specified sampling cycle (e.g., 20 (msec)) in the period during which the answer image P3 is displayed. In response to detection of the position data on the gaze point P of the subject, the determination unit 34 determines in which area the gaze point P of the subject is present among the specific area X1 and the comparison areas X2 to X4 and outputs determination data. Therefore, the determination unit 34 outputs the determination data at each determination cycle that is the same as the above-described sampling cycle.

The calculation unit 35 calculates the evaluation parameters indicating the course of movement of the gaze point P during the display period based on the determination data. The calculation unit 35 calculates, as the evaluation parameters, for example, the presence time data, the movement number data, the last area data, and the arrival time data.

The presence time data indicates the presence time during which the gaze point P is present in the specific area X1. According to the present embodiment, it may be assumed that the greater the number of times the determination unit 34 determines that the gaze point P is present in the specific area X1, the longer the presence time during which the gaze point P is present in the specific area X1. Therefore, the presence time data may be the number of times the determination unit 34 determines that the gaze point P is present in the specific area X1. That is, the calculation unit 35 may use a count value NX1 of the counter as the presence time data.

The movement number data indicates the number of times the position of the gaze point P moves among the comparison areas X2 to X4 before the gaze point P first arrives at the specific area X1. Therefore, the calculation unit 35 may count the number of times the gaze point P has moved among the specific area X1 and the comparison areas X2 to X4 and use the count result before the gaze point P arrives at the specific area X1 as the movement number data.

The last area data indicates the area where the gaze point P is last present among the specific area X1 and the comparison areas X2 to X4, i.e., the last area that is gazed at as an answer by the subject. Each time the gaze point P is detected, the calculation unit 35 updates the area where the gaze point P is present to thereby obtain the detection result at the end time of the display of the answer image P3 as the last area data.

The arrival time data indicates the time from the start time of display of the answer image P3 until the arrival time when the gaze point P first arrives at the specific area X1. Therefore, the calculation unit 35 uses a timer T to measure the elapsed time from the start of display, and when the gaze point P first arrives at the specific area X1, sets a flag value to 1 and detects the measured value of the timer T to thereby obtain the detection result of the timer T as the arrival time data.

The evaluation unit 36 calculates an evaluation value based on the presence time data, the movement number data, the last area data, and the arrival time data and obtains evaluation data based on the evaluation value. For example, the last area data has a data value D1, the presence time data has a data value D2, the arrival time data has a data value D3, and the movement number data has a data value D4. The data value D1 of the last area data is 1 when the final gaze point P of the subject is present in the specific area X1 (that is, when the answer is correct) and is 0 when the final gaze point P of the subject is not present in the specific area X1 (that is, when the answer is incorrect). The data value D2 of the presence time data is the number of seconds in which the gaze point P is present in the specific area X1. An upper limit, which is the number of seconds shorter than the display period, may be set for the data value D2. The data value D3 of the arrival time data is the reciprocal of the arrival time, e.g., 1/(arrival time)/10. The value “10” is the coefficient for setting an arrival time evaluation value to 1 or less when the minimum value of the arrival time is 0.1 seconds. The count value is used as it is as the data value D4 of the movement number data. An upper limit may be set as appropriate for the data value D4.

In this case, an evaluation value ANS1 may be represented as, for example,

ANS1=D1·K1+D2·K2+D3·K3+D4·K4

where K1 to K4 are constants for weighting. The constants K1 to K4 may be set as appropriate.

In a case where the data value D1 of the last area data is 1, the evaluation value ANS1 represented by the above equation becomes large when the data value D2 of the presence time data is large, when the data value D3 of the arrival time data is large, and when the data value D4 of the movement number data is large. That is, the evaluation value ANS1 becomes larger when the final gaze point P is present in the specific area X1, the presence time of the gaze point P in the specific area X1 is longer, the arrival time from when the display period is started to when the gaze point P arrives at the specific area X1 is shorter, and the number of times the gaze point P moves among the areas is larger.

In a case where the data value D1 of the last area data is 0, the evaluation value ANS1 becomes small when the data value D2 of the presence time data is small, when the data value D3 of the arrival time data is small, and when the data value D4 of the movement number data is small. That is, the evaluation value ANS1 becomes smaller when the final gaze point P is not present in the specific area X1, the presence time of the gaze point P in the specific area X1 is shorter, the arrival time from when the display period is started to when the gaze point P arrives at the specific area X1 is longer, and the number of times the gaze point P moves among the areas is smaller.

Therefore, the evaluation unit 36 may determine whether the evaluation value ANS1 is equal to or more than a predetermined value to thereby obtain the evaluation data. For example, when the evaluation value ANS1 is equal to or more than the predetermined value, the evaluation may indicate that the subject is unlikely to be a person having cognitive dysfunction and brain dysfunction. When the evaluation value ANS1 is less than the predetermined value, the evaluation may indicate that the subject is highly likely to be a person having cognitive dysfunction and brain dysfunction.

The evaluation unit 36 may store the evaluation value ANS1 in the storage unit 38. For example, the evaluation values ANS1 for the same subject may be cumulatively stored to make a comparative evaluation using the past evaluation value. For example, when the evaluation value ANS1 is higher than the past evaluation value, the evaluation may indicate that the brain function has improved as compared with the previous evaluation. When the cumulative value of the evaluation values ANS1 gradually increases, for example, the evaluation may indicate that the brain function has been gradually improved.

The evaluation unit 36 may make an evaluation by individually using the presence time data, the movement number data, the last area data, and the arrival time data or by combining two or more of the presence time data, the movement number data, the last area data, and the arrival time data. For example, when the gaze point P accidentally arrives at the specific area X1 while the subject looks at many objects, the data value D4 of the movement number data becomes small. In this case, the evaluation may be made together with the data value D2 of the above-described presence time data. For example, when the presence time is long, even though the number of movements is small, the evaluation may indicate that the subject can gaze at the specific area X1, which is a correct answer. When the number of movements is small and the presence time is also short, the evaluation may indicate that the gaze point P accidentally passed through the specific area X1.

When the number of movements is small and the last area is the specific area X1, the evaluation may indicate that, for example, the specific area X1, which is a correct answer, was reached with the small number of movements of the gaze point P. When the number of movements described above is small and when the last area is not the specific area X1, the evaluation may indicate that, for example, the gaze point P accidentally passed through the specific area X1. Therefore, the evaluation using the evaluation parameters makes it possible to obtain the evaluation data based on the course of movement of the gaze point P, and thus the effect of accidentalness may be reduced.

According to the present embodiment, when the evaluation unit 36 outputs the evaluation data, the input/output control unit 37 may cause the output device 40 to output, based on the evaluation data, text data such as “the subject is unlikely to be a person having cognitive dysfunction and brain dysfunction” or text data such as “the subject is likely to be a person having cognitive dysfunction and brain dysfunction”. When the evaluation value ANS1 for the same subject is higher than the past evaluation value ANS1, the input/output control unit 37 may cause the output device 40 to output text data such as “brain function has improved”.

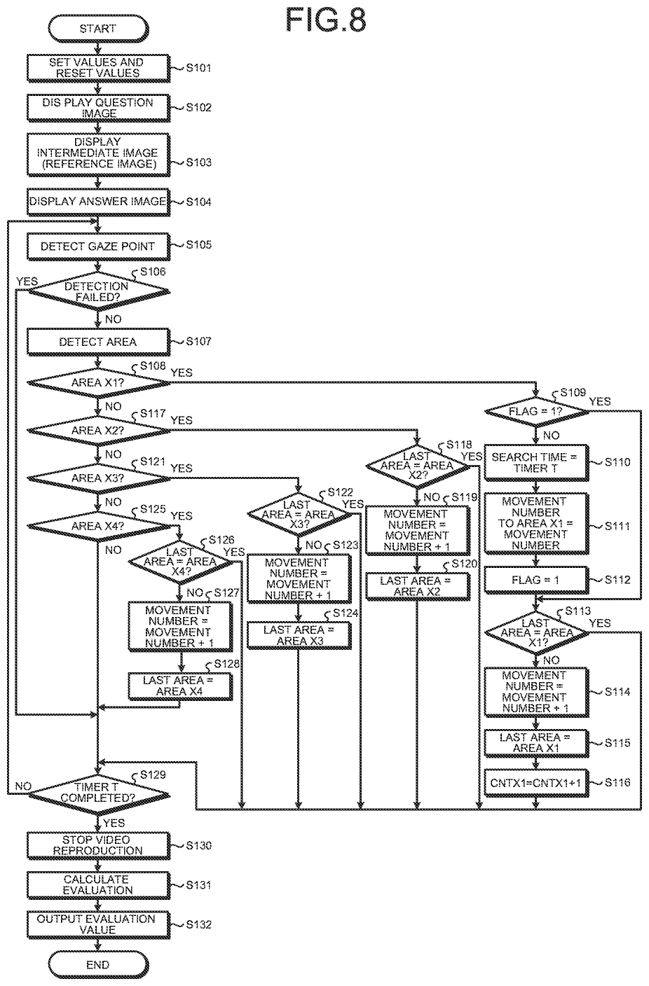

Next, an example of the evaluation method according to the present embodiment is described with reference to FIG. 8. FIG. 8 is a flowchart illustrating an example of the evaluation method according to the present embodiment. According to the present embodiment, the calculation unit 35 executes the following setting and resetting (Step S101). First, the calculation unit 35 sets display times T1, T2, and T3 for displaying the question image P1, the intermediate image P2, and the answer image P3, respectively. The calculation unit 35 resets the timer T and the count value NX1 of the counter and resets the flag value to 0. The display control unit 31 may set transmissivity α of the reference image R1 illustrated in the intermediate image P2.

After executing the above setting and resetting, the display control unit 31 displays the question image P1 on the display unit 11 (Step S102). The display control unit 31 displays the intermediate image P2 on the display unit 11 after the elapse of the display time T1 set at Step S101 from the display of the question image P1 (Step S103). A process may be performed to superimpose the reference image R1 on the question image P1. The display control unit 31 displays the answer image P3 after the elapse of the display time T2 set at Step S101 from the display of the intermediate image P2 (Step S104). When the answer image P3 is displayed, the area setting unit 33 sets the specific area X1 and the comparison areas X2 to X4 in the answer image P3.

The gaze point detection unit 32 detects the position data on the gaze point P of the subject on the display unit 11 at each specified sampling cycle (e.g., 20 (msec)) while the subject looks at the image displayed on the display unit 11 (Step S105). When the position data is detected (No at Step S106), the determination unit 34 determines the area where the gaze point P is present, based on the position data (Step S107). When no position data is detected (Yes at Step S106), the process at Step S129 and the subsequent steps described below is performed.

When it is determined that the gaze point P is present in the specific area X1 (Yes at Step S108), the calculation unit 35 determines whether a flag value F is 1, i.e., the gaze point P arrived at the specific area X1 for the first time (1: arrived, 0: not arrived) (Step S109). When the flag value F is 1 (Yes at Step S109), the calculation unit 35 skips the following Steps S110 to S112 and performs the process at Step S113 described below.

When the flag value F is not 1, i.e., when the gaze point P arrived at the specific area X1 for the first time (No at Step S109), the calculation unit 35 extracts the measurement result of the timer T as the arrival time data (Step S110). The calculation unit 35 stores, in the storage unit 38, the movement number data indicating the number of times the gaze point P has moved among the areas before arriving at the specific area X1 (Step S111). Subsequently, the calculation unit 35 changes the flag value to 1 (Step S112).

Subsequently, the calculation unit 35 determines whether the area where the gaze point P is present during the most recent detection, i.e., the last area, is the specific area X1 (Step S113). When it is determined that the last area is the specific area X1 (Yes at Step S113), the calculation unit 35 skips the following Steps S114 to S116 and performs the process at Step S129 described below. When it is determined that the last area is not the specific area X1 (No at Step S113), the calculation unit 35 increments by one the cumulative number indicating the number of times the gaze point P has moved among the areas (Step S114) and changes the last area to the specific area X1 (Step S115). The calculation unit 35 increments by one the count value NX1 indicating the presence time data in the specific area X1 (Step S116). Subsequently, the calculation unit 35 performs the process at Step S129 and the subsequent steps described below.

When it is determined that the gaze point P is not present in the specific area X1 (No at Step S108), the calculation unit 35 determines whether the gaze point P is present in the comparison area X2 (Step S117). When it is determined that the gaze point P is present in the comparison area X2 (Yes at Step S117), the calculation unit 35 determines whether the area where the gaze point P is present during the most recent detection, i.e., the last area, is the comparison area X2 (Step S118). When it is determined that the last area is the comparison area X2 (Yes at Step S118), the calculation unit 35 skips the following Steps S119 and S120 and performs the process at Step S129 described below. When it is determined that the last area is not the comparison area X2 (No at Step S118), the calculation unit 35 increments by one the cumulative number indicating the number of times the gaze point P has moved among the areas (Step S119) and changes the last area to the comparison area X2 (Step S120). Subsequently, the calculation unit 35 performs the process at Step S129 and the subsequent steps described below.

When it is determined that the gaze point P is not present in the comparison area X2 (No at Step S117), the calculation unit 35 determines whether the gaze point P is present in the comparison area X3 (Step S121). When it is determined that the gaze point P is present in the comparison area X3 (Yes at Step S121), the calculation unit 35 determines whether the area where the gaze point P is present during the most recent detection, i.e., the last area, is the comparison area X3 (Step S122). When it is determined that the last area is the comparison area X3 (Yes at Step S122), the calculation unit 35 skips the following Steps S123 and S124 and performs the process at Step S129 described below. When it is determined that the last area is not the comparison area X3 (No at Step S122), the calculation unit 35 increments by one the cumulative number indicating the number of times the gaze point P has moved among the areas (Step S123) and changes the last area to the comparison area X3 (Step S124). Subsequently, the calculation unit 35 performs the process at Step S129 and the subsequent steps described below.

When it is determined that the gaze point P is not present in the comparison area X3 (No at Step S121), the calculation unit 35 determines whether the gaze point P is present in the comparison area X4 (Step S125). When it is determined that the gaze point P is present in the comparison area X4 (Yes at Step S125), the calculation unit 35 determines whether the area where the gaze point P is present during the most recent detection, i.e., the last area, is the comparison area X4 (Step S126). When it is determined that the gaze point P is not present in the comparison area X4 (No at Step S125), the process at Step S129 described below is performed. When it is determined that the last area is the comparison area X4 (Yes at Step S126), the calculation unit 35 skips the following Steps S127 and S128 and performs the process at Step S129 described below. When it is determined that the last area is not the comparison area X4 (No at Step S126), the calculation unit 35 increments by one the cumulative number indicating the number of times the gaze point P has moved among the areas (Step S127) and changes the last area to the comparison area X4 (Step S128). Subsequently, the calculation unit 35 performs the process at Step S129 and the subsequent steps described below.

Subsequently, the calculation unit 35 determines whether the display time T3 of the answer image P3 has elapsed based on the detection result of the timer T (Step S129). When it is determined that the display time T3 of the answer image P3 has not elapsed (No at Step S129), the process at Step S105 and the subsequent steps described above is repeatedly performed.

When the calculation unit 35 determines that the display time T3 of the answer image P3 has elapsed (Yes at Step S129), the display control unit 31 stops the play of the video (Step S130). After the paly of the video is stopped, the evaluation unit 36 calculates the evaluation value ANS1 based on the presence time data, the movement number data, the last area data, and the arrival time data obtained from the above processing results (Step S131) and obtains the evaluation data based on the evaluation value ANS1. Subsequently, the input/output control unit 37 outputs the evaluation data obtained by the evaluation unit 36 (Step S132).

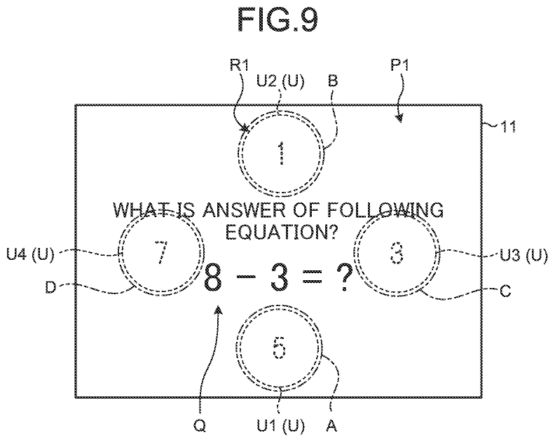

When the intermediate image P2 is displayed on the display unit 11, the subject may be evaluated by using the first object U1 and the second objects U2 to U4 included in the intermediate image P2 (the reference image R1). FIG. 9 is a diagram illustrating another example of the intermediate image displayed on the display unit 11. As illustrated in FIG. 9, the display control unit 31 displays, on the display unit 11, the intermediate image P2 including the question image P1 and the reference image R1 after displaying the question image P1 for a predetermined time. In this case, the area setting unit 33 sets a first reference area A corresponding to the first object U1 during the period of displaying the intermediate image P2 (the reference image R1). The area setting unit 33 sets second reference areas B, C, and D corresponding to the second objects U2 to U4. The reference image R1 is described below as an example of the reference image included in the intermediate image P2; however, the same description is applicable to a case where the reference image R2 is included.

The area setting unit 33 may set the reference areas A to D in the respective areas including at least parts of the first object U1 and the second objects U2 to U4. According to the present embodiment, the area setting unit 33 sets the first reference area A in the circular area including the first object U1 and sets the second reference areas B to D in the circular areas including the second objects U2 to U4. In this manner, the area setting unit 33 may set the reference areas A to D corresponding to the reference image R1.

The gaze point detection unit 32 detects the position data on the gaze point P of the subject at each specified sampling cycle (e.g., 20 (msec)) during the period of displaying the intermediate image P2. In response to detection of the position data on the gaze point P of the subject, the determination unit 34 determines in which reference area the gaze point P of the subject is present among the first reference area A and the second reference areas B to D and outputs determination data. Thus, the determination unit 34 outputs determination data at each determination cycle that is the same as the above-described sampling cycle.

Based on the determination data, the calculation unit 35 calculates the evaluation parameter indicating the course of movement of the gaze point P during the period of displaying the intermediate image P2 in the same manner as described above. The calculation unit 35 calculates, for example, the presence time data, the movement number data, the last area data, and the arrival time data as evaluation parameters.

The presence time data indicates the presence time during which the gaze point P is present in the first reference area A. The presence time data may be the number of times the determination unit 34 determines that the gaze point P is present in the first reference area A. Specifically, the calculation unit 35 may use count values NA, NB, NC, and ND of counters as the presence time data.

The movement number data indicates the number of times the position of the gaze point P moves among the second reference areas B to D before the gaze point P first arrives at the first reference area A. The calculation unit 35 may count the number of times the gaze point P moves among the first reference area A and the second reference areas B to D and use the count result before the gaze point P arrives at the first reference area A as the movement number data.

The last area data indicates the last area where the gaze point P is present among the first reference area A and the second reference areas B to D, i.e., the last area that is gazed at as an answer by the subject. Each time the gaze point P is detected, the calculation unit 35 updates the area where the gaze point P is present to thereby obtain the detection result at the end time of the display of the answer image P3 as the last area data.

The arrival time data indicates the time from the start time of display of the intermediate image P2 until the arrival time when the gaze point P first arrives at the first reference area A. The calculation unit 35 uses the timer T to measure the elapsed time from the start of display, and when the gaze point P first arrives at the first reference area A, detects the measured value of the timer T to thereby obtain the detection result of the timer T as the arrival time data.

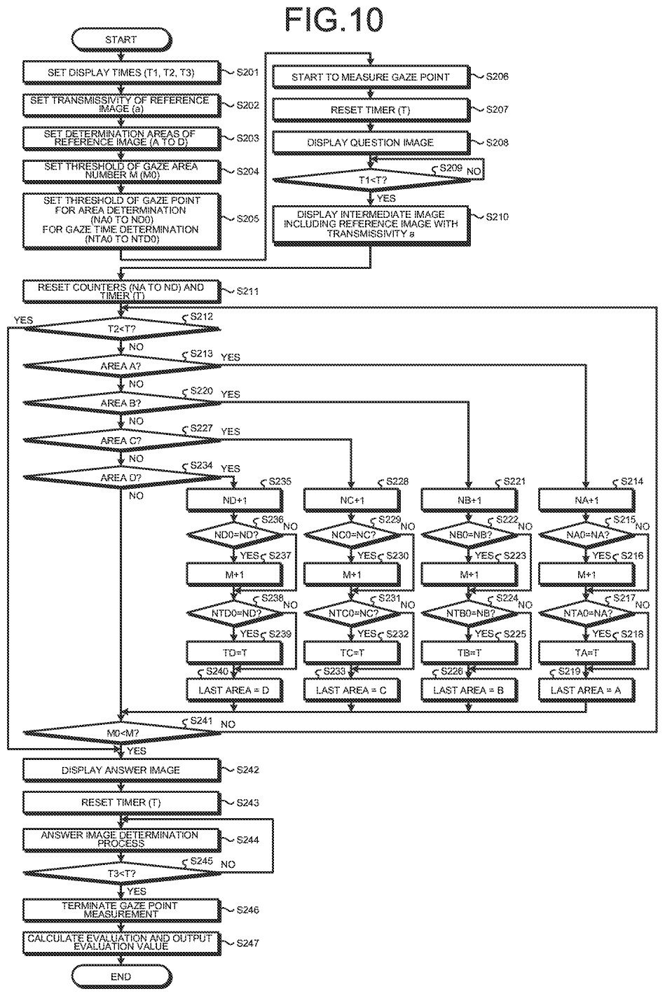

FIG. 10 is a flowchart illustrating another example of the evaluation method according to the present embodiment. As illustrated in FIG. 10, first, the display times (predetermined times) T1, T2, and T3 for displaying the question image P1, the intermediate image P2, and the answer image P3 are set (Step S201), and the transmissivity α of the reference image R1 to be displayed on the intermediate image P2 is set (Step S202). The first reference area A and the second reference areas B to D in the intermediate image P2 are set (Step S203).

For the first reference area A and the second reference areas B to D, a threshold MO is set for a gaze area number M indicating how many areas the subject has gazed at (Step S204). In the example of FIG. 9, as there are four areas (A to D), the threshold MO is set in the range from 0 to 4. Thresholds described below are set for the gaze point (Step S205). First, numbers NA0 to ND0 of gaze points needed to determine that the first reference area A and the second reference areas B to D have been gazed at are set, respectively. When the obtained gaze points are equal to or more than the numbers NA0 to ND0 respectively set for the first reference area A and the second reference areas B to D, it is determined that the corresponding area has been gazed at. Gaze point numbers NTA0 to NTD0 used to determine times TA to TD from when the intermediate image P2 is displayed until when each area (the first reference area A and the second reference areas B to D) in the reference image R1 is recognized are also set.

After making the above-described settings, the gaze point detection unit 32 starts to measure the gaze point (Step S206). The calculation unit 35 resets the timer T, which measures the elapsed time, and starts timing (Step S207). The display control unit 31 displays the question image P1 on the display unit 11 (Step S208). After starting to display the question image P1, the display control unit 31 waits until the display time T1 set at Step S201 has elapsed (Step S209).

After the display time T1 has elapsed (Yes at Step 209), the display control unit 31 displays, on the display unit 11, the intermediate image P2 including the reference image R1 having the transmissivity α set at Step S202 (Step S210). At this point, the area setting unit 33 sets the first reference area A corresponding to the first object U1 in the reference image R1 and the second reference areas B to D corresponding to the second objects U2 to U4. At the same time as the start of display of the intermediate image P2, the count values NA to ND are reset in the counters that count the gaze point in the first reference area A and the second reference areas B to D, and the timer T that measures the elapsed time is reset and is started for timing (Step S211). Subsequently, the display control unit 31 waits until the display time T2 set at Step S202 has elapsed (Step S212).

After the display time T2 has elapsed (Yes at Step S212), the display control unit 31 displays the answer image P3 on the display unit 11 (Step S242). When the display time T2 has not elapsed (No at Step S212), the area determination described below is performed.

When it is determined that the gaze point P is present in the first reference area A (Yes at Step S213), the calculation unit 35 increments by one the count value NA for the first reference area A (Step S214). When the count value NA has reached the number NA0 (Yes at Step S215), the gaze area number M is incremented by 1 (Step S216). When the count value NA has reached the gaze point number NTA0 (Yes at Step S217), the value of the timer T is the time TA it took to recognize the first reference area A (Step S218). Subsequently, the last area is changed to the first reference area A (Step S219).

When it is determined that the gaze point P is not present in the first reference area A (No at Step S213), the same process as that in Steps S213 to S219 is performed for the gaze point P in each of the second reference areas B to D. Specifically, the process at Steps S220 to S226 is performed for the second reference area B. The process at Steps S227 to S233 is performed for the second reference area C. The process at Steps S234 to S240 is performed for the second reference area D.

After the process at Steps S219, S226, S233, S240, or No at Step S234, the calculation unit 35 determines whether the gaze area number M of the subject has reached the threshold MO set at Step S205 (Step S241). When the threshold MO has not been reached (No at Step S241), the process at Step S212 and the subsequent steps is repeatedly performed. When the threshold MO has been reached (Yes at Step S241), the display control unit 31 displays the answer image P3 on the display unit 11 (Step S242). Subsequently, the calculation unit 35 resets the timer T (Step S243) and performs the same process as the above-described determination process (see Steps S105 to S128 illustrated in FIG. 8) for the answer image P3 described in FIG. 8 (Step S244). Then, the calculation unit 35 determines whether the count value of the timer T has reached the display time T3 set at Step S201 (Step S245). When the display time T3 has not been reached (No at Step S245), the calculation unit 35 repeatedly performs the process at Step S244. When the display time T3 has been reached (Yes at Step S245), the gaze point detection unit 32 terminates the measurement of the gaze point P (Step S246). Subsequently, the evaluation unit 36 performs an evaluation calculation (Step S247).

The evaluation unit 36 obtains the evaluation value based on the presence time data, the movement number data, the last area data, and the arrival time data and obtains the evaluation data based on the evaluation value. The evaluation by the evaluation unit 36 may be the same as the evaluation for the answer image P3 described above. Here, for example, the last area data has a data value D5, the arrival time data has a data value D6, the presence time data has a data value D7, and the movement number data has a data value D8. The data value D5 of the last area data is 1 when the final gaze point P of the subject is present in the first reference area A (that is, when the answer is correct) and is 0 when the final gaze point P of the subject is not present in the first reference area A (that is, when the answer is incorrect). The data value D6 of the arrival time data is the reciprocal of the arrival time TA (e.g., [1/(arrival time)]/10) (10: the coefficient for setting an arrival time evaluation value to 1 or less when the minimum value of the arrival time is 0.1 seconds). The data value D7 of the presence time data may be represented by using the ratio (NA/NA0) (the maximum value is 1.0) at which the first reference area A has been gazed at. The data value D8 of the movement number data may be represented by using the ratio (M/M0) obtained by dividing the gaze area number M of the subject by the threshold M0.

In this case, an evaluation value ANS2 may be represented as, for example,

ANS2=D5·K5+D6·K6+D7·K7+D8·K8

where K5 to K8 are constants for weighting. The constants K5 to K8 may be set as appropriate.

In a case where the data value D5 of the last area data is 1, the evaluation value ANS2 represented by the above equation becomes large w, when the data value D6 of the arrival time data is large, when the data value D7 of the presence time data is large, and when the data value D8 of the movement number data is large. That is, the evaluation value ANS2 becomes larger when the final gaze point P is present in the first reference area A, the arrival time from when the display of the reference image R1 is started to when the gaze point P arrives at the first reference area A is shorter, the presence time of the gaze point P in the first reference area A is longer, and the number of times the gaze point P moves among the areas is larger.

In a case where the data value D5 of the last area data is 0, the evaluation value ANS2 becomes small, when the data value D6 of the arrival time data is small, when the data value D7 of the presence time data is small, and when the data value D8 of the movement number data is small. That is, the evaluation value ANS2 becomes smaller when the final gaze point P is present in the second reference areas B to D, the arrival time from when the display of the reference image R1 is started to when the gaze point P arrives at the first reference area A is longer (or no arrival), the presence time of the gaze point P in the first reference area A is shorter (or no presence), and the number of times the gaze point P moves among the areas is smaller.

When the evaluation value ANS2 is large, it may be determined that the reference image R1 was quickly recognized, the content of the question information Q was accurately understood, and then the correct answer (the first reference area A) was gazed at. Conversely, when the evaluation value ANS2 is small, it may be determined that the reference image R1 was not quickly recognized, the content of the question information Q was not accurately understood, or the correct answer (the first reference area A) was not gazed at.

Therefore, the evaluation unit 36 may determine whether the evaluation value ANS2 is equal to or more than a predetermined value to thereby obtain the evaluation data. For example, when the evaluation value ANS2 is equal to or more than the predetermined value, the evaluation may indicate that the subject is unlikely to be a person having cognitive dysfunction and brain dysfunction. When the evaluation value ANS2 is less than the predetermined value, the evaluation may indicate that the subject is highly likely to be a person having cognitive dysfunction and brain dysfunction.

The evaluation unit 36 may store the evaluation value ANS2 in the storage unit 38 in the same manner as described above. For example, the evaluation values ANS2 for the same subject may be cumulatively stored to make a comparative evaluation using the past evaluation value. For example, when the evaluation value ANS2 is higher than the past evaluation value, the evaluation may indicate that the brain function has improved as compared with the previous evaluation. When the cumulative value of the evaluation values ANS2 gradually increases, for example, the evaluation may indicate that the brain function has been gradually improved.

The evaluation unit 36 may make an evaluation by individually using the presence time data, the movement number data, the last area data, and the arrival time data or by combining two or more of the presence time data, the movement number data, the last area data, and the arrival time data. For example, when the gaze point P accidentally arrives at the first reference area A while the subject looks at many objects, the data value D8 of the movement number data becomes small. In this case, the evaluation may be made together with the data value D7 of the above-described presence time data. For example, when the presence time is long, even though the number of movements is small, the evaluation may indicate that the subject can gaze at the first reference area A, which is a correct answer. When the number of movements is small and the presence time is also short, the evaluation may indicate that the gaze point P accidentally passed through the first reference area A.

When the number of movements is small and the last area is the first reference area A, the evaluation may indicate that, for example, the first reference area A, which is a correct answer, was reached with the small number of movements of the gaze point P. When the number of movements described above is small and when the last area is not the first reference area A, the evaluation may indicate that, for example, the gaze point P accidentally passed through the first reference area A. Therefore, the evaluation using the evaluation parameters makes it possible to obtain the evaluation data based on the course of movement of the gaze point P, and thus the effect of accidentalness may be reduced.

The evaluation unit 36 may determine a final evaluation value ANS by using the evaluation value ANS1 in the answer image P3 and the evaluation value ANS2 in the question image P1 described above. In this case, the final evaluation value ANS may be represented as, for example,

ANS=ANS1·K9+ANS2·K10

where K9 and K10 are constants for weighting. The constants K9 and K10 may be set as appropriate.

When the evaluation value ANS1 is large and the evaluation value ANS2 is large, the evaluation may indicate that there is no risk in, for example, the cognitive ability, the comprehension ability, and the processing ability in whole for the question information Q.

When the evaluation value ANS1 is large and the evaluation value ANS2 is small, the evaluation may indicate that there is no risk in, for example, the comprehension ability and the processing ability for the question information Q but there is a risk in the cognitive ability for the question information Q.

When the evaluation value ANS1 is small and the evaluation value ANS2 is small, the evaluation may indicate that there are risks in, for example, the cognitive ability, the comprehension ability, and the processing ability in whole for the question information Q.

As described above, the evaluation device 100 according to the present embodiment includes: the display unit 11; the gaze point detection unit 32 that detects the position of the gaze point of the subject on the display unit 11; the display control unit 31 that, after displaying the question image including the question information for the subject on the display unit 11, displays, on the display unit 11, the answer image including the specific object that is a correct answer to the question information and one or more comparison objects different from the specific object, and when the question image is displayed on the display unit 11, displays, on the display unit 11, the reference image illustrating the positional relationship between the specific object and the one or more comparison objects in the answer image; the area setting unit 33 that sets, on the display unit 11, the specific area corresponding to the specific object and one or more comparison areas corresponding to the one or more comparison object; the determination unit 34 that determines, at each specified determination cycle, in which area the gaze point is present among the specific area and the one or more comparison areas, based on the position of the gaze point; the calculation unit 35 that calculates the evaluation parameter based on the determination result of the determination unit 34; and the evaluation unit 36 that obtains the evaluation data on the subject based on the evaluation parameter.

An evaluation method according to the present embodiment includes: detecting the position of the gaze point of the subject on the display unit 11; after displaying the question image including the question information for the subject on the display unit 11, displaying, on the display unit 11, the answer image including the specific object that is a correct answer to the question information and one or more comparison objects different from the specific object, and when the question image is displayed on the display unit 11, displaying, on the display unit 11, the reference image illustrating the positional relationship between the specific object and the one or more comparison objects in the answer image; setting, on the display unit 11, the specific area corresponding to the specific object and one or more comparison areas corresponding to the one or more comparison objects; the determination unit 34 that determines, at each specified determination cycle, in which area the gaze point is present among the specific area and the one or more comparison areas; calculating the evaluation parameter based on the determination result of the determination unit 34, based on the position of the gaze point; and obtaining the evaluation data on the subject based on the evaluation parameter.

An evaluation program according to the present embodiment causes a computer to execute: detecting the position of the gaze point of the subject on the display unit 11; after displaying the question image including the question information for the subject on the display unit 11, displaying, on the display unit 11, the answer image including the specific object that is a correct answer to the question information and one or more comparison objects different from the specific object, and when the question image is displayed on the display unit 11, displaying, on the display unit 11, the reference image illustrating the positional relationship between the specific object and the one or more comparison objects in the answer image; setting, on the display unit 11, the specific area corresponding to the specific object and one or more comparison areas corresponding to the one or more comparison objects; determining, at each specified determination cycle, in which area the gaze point is present among the specific area and the one or more comparison areas, based on the position of the gaze point; calculating the evaluation parameter based on the determination result of the determination unit 34; and obtaining the evaluation data on the subject based on the evaluation parameter.

According to the present embodiment, the subject gazes at the reference image R in the question image P1 before the answer image P3 is displayed so as to understand the arrangement of the specific object M1 and the comparison objects M2 to M4. Accordingly, after the answer image P3 is displayed, the subject may quickly gaze at the specific object M1 that is a correct answer to the question information Q. Furthermore, the evaluation using the evaluation parameters makes it possible to obtain the evaluation data based on the course of movement of the gaze point P, and thus the effect of accidentalness may be reduced.

In the evaluation device 100 according to the present embodiment, the area setting unit 33 sets, on the display unit 11, the reference areas A to D corresponding to the reference image R1, and the determination unit 34 determines in which area the gaze point P is present among the reference areas A to D based on the position of the gaze point P. Thus, the evaluation including the evaluation parameter for the reference image R1 may be performed.

In the evaluation device 100 according to the present embodiment, the reference image R1 includes the first object U1 corresponding to the specific object M1 and the second objects U2 to U4 corresponding to the comparison objects M2 to M4, and the area setting unit 33 sets, as the reference areas, the first reference area A corresponding to the first object U1 in the reference image R1 and the second reference areas B to D corresponding to the second objects U2 to U4 in the reference image R1. Thus, the evaluation may be obtained at a stage before the answer image P3 is displayed.

In the evaluation device 100 according to the present embodiment, the evaluation parameter includes at least one set of data among the arrival time data indicating the time until the arrival time when the gaze point P first arrives at the first reference area A, the movement number data indicating the number of times the position of the gaze point P moves among the second reference areas B to D before the gaze point P first arrives at the first reference area A, and the presence time data indicating the presence time during which the gaze point is present in the first reference area A in a display period of the reference image R1 and the last area data indicating the last area where the gaze point is present in the display period among the first reference area A and the second reference areas B to D. Therefore, it is possible to obtain a highly accurate evaluation without removing accidentalness.

In the evaluation device 100 according to the present embodiment, the reference image is an image (R1) obtained by changing the transmissivity of the answer image P3 or an image (R2) obtained by reducing the answer image P3. By using the answer image P3 as a reference image, the positional relationship between the specific object M1 and the comparison objects M2 to M4 in the answer image P3 may be easily understood.

In the evaluation device 100 according to the present embodiment, the display control unit 31 displays the reference image R1 after the elapse of a predetermined time from start of display of the question image P1. Thus, it is possible to give the subject some time to consider the content of the question information Q, and it is possible to avoid confusion caused to the subject.

The technical scope of the present disclosure is not limited to the above-described embodiment, and changes may be made as appropriate without departing from the spirit of the present disclosure. For example, in the description according to the above embodiment, the display control unit 31 displays the reference image R1 after the elapse of the predetermined time from the start of display of the question image P1, but this is not a limitation. For example, the display control unit 31 may display the reference image R1 at the same time as the start of display of the question image P1. The display control unit 31 may display the reference image R1 before the question image P1 is displayed.

With the evaluation device, the evaluation method, and the evaluation program according to the present disclosure, it is possible to accurately evaluate cognitive dysfunction and brain dysfunction.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An evaluation device comprising:
a display unit;
a gaze point detection unit configured to detect a position of a gaze point of a subject on the display unit;
a display control unit configured to, after displaying a question image including question information for the subject on the display unit, display, on the display unit, an answer image including a specific object that is a correct answer to the question information and one or more comparison objects different from the specific object, and when the question image is displayed on the display unit, display, on the display unit, a reference image illustrating a positional relationship between the specific object and the one or more comparison objects in the answer image;
an area setting unit configured to set, on the display unit, a specific area corresponding to the specific object and one or more comparison areas corresponding to the one or more comparison objects;
a determination unit configured to determine, at each specified determination cycle, in which area the gaze point is present among the specific area and the one or more comparison areas, based on a position of the gaze point;
a calculation unit configured to calculate an evaluation parameter based on a determination result of the determination unit; and
an evaluation unit configured to obtain evaluation data on the subject based on the evaluation parameter.

2. The evaluation device according to claim 1, wherein
the area setting unit is configured to set, on the display unit, reference areas corresponding to the reference image, and
the determination unit is configured to determine in which reference area the gaze point is present among the reference areas, based on the position of the gaze point.

3. The evaluation device according to claim 2, wherein
the reference image includes a first object corresponding to the specific object and one or more second objects corresponding to the one or more comparison objects, and
the area setting unit is configured to set, as the reference areas, a first reference area corresponding to the first object in the reference image and one or more second reference areas corresponding to the one or more second objects in the reference image.

4. The evaluation device according to claim 3, wherein the evaluation parameter includes
at least one piece of data among arrival time data indicating a time until an arrival time when the gaze point first arrives at the first reference area, movement number data indicating a number of times the position of the gaze point moves between the second reference areas before the gaze point first arrives at the first reference area, and presence time data indicating a presence time during which the gaze point is present in the first reference area in a display period of the reference image, and
last area data indicating an area where the gaze point is last present in the display period among the first reference area and the second reference areas.

5. The evaluation device according to claim 1, wherein the reference image is an image obtained by changing transmissivity of the answer image or an image obtained by reducing the answer image.

6. The evaluation device according to claim 1, wherein the display control unit is configured to display the reference image after elapse of a predetermined time from start of display of the question image.

7. An evaluation method comprising:
detecting a position of a gaze point of a subject on a display unit;
after displaying a question image including question information for the subject on the display unit, displaying, on the display unit, an answer image including a specific object that is a correct answer to the question information and one or more comparison objects different from the specific object, and when the question image is displayed on the display unit, displaying, on the display unit, a reference image illustrating a positional relationship between the specific object and the one or more comparison objects in the answer image;
setting, on the display unit, a specific area corresponding to the specific object and one or more comparison areas corresponding to the one or more comparison objects;
determining, at each specified determination cycle, in which area the gaze point is present among the specific area and the one or more comparison areas, based on a position of the gaze point;
calculating an evaluation parameter based on a determination result obtained at the determining; and
obtaining evaluation data on the subject based on the evaluation parameter.

8. A non-transitory computer-readable recording medium containing a computer program causing a computer to execute:
detecting a position of a gaze point of a subject on a display unit;
after displaying a question image including question information for the subject on the display unit, displaying, on the display unit, an answer image including a specific object that is a correct answer to the question information and one or more comparison objects different from the specific object, and when the question image is displayed on the display unit, displaying, on the display unit, a reference image illustrating a positional relationship between the specific object and the one or more comparison objects in the answer image;
setting, on the display unit, a specific area corresponding to the specific object and one or more comparison areas corresponding to the one or more comparison objects;
determining, at each specified determination cycle, in which area the gaze point is present among the specific area and the one or more comparison areas, based on a position of the gaze point;
calculating an evaluation parameter based on a determination result obtained at the determining; and
obtaining evaluation data on the subject based on the evaluation parameter.
